(19) United States
(12) Patent Application Publication
(10) Pub. No.: US 2011/0021657 A1
(43) Pub. Date: Jan. 27, 2011

(54) HYDROPHILIC POLYURETHANE SOLUTIONS

(75) Inventor: Jürgen Köcher, Langenfeld (DE)

Correspondence Address: CONNOLLY BOVE LODGE & HUTZ, LLP, P O BOX 2207, WILMINGTON, DE, 19899, US

(73) Assignee: Bayer MaterialScience AG, Leverkusan (DE)

(21) Appl. No.: 12/933,473

(22) PCT Filed: Mar. 16, 2009
(86) PCT No.: PCT/EP2009/001899

§ 371 Date: Sep. 20, 2010

(30) Foreign Application Priority Data
Mar. 20, 2008 (EP) 08153055.2

(51) Int. Cl. A61F 2/00; C08L 75/04

(52) U.S. Cl. 523/113; 524/590

(57) ABSTRACT

The present invention relates to a coating compound comprising at least one polyurethane urea terminated with a copolymerized unit of polyethylene oxide and polypropylene oxide.

The present invention relates to a coating composition in the form of a polyurethane solution that can be used for producing hydrophilic coatings. Further subject matter of the present invention is a process for preparing such a coating composition, and the use of the coating composition, more particularly for the coating of medical devices.

The utilization of medical devices, such as of catheters, can be improved greatly through the equipping thereof with hydrophilic surfaces. The insertion and displacement of urinary or blood vessel catheters is made easier by the fact that hydrophilic surfaces in contact with blood or urine adsorb a water film. This reduces the friction between the catheter surface and the vessel walls, so making the catheter easier to insert and move. Direct watering of the devices prior to the intervention can also be carried out, in order to reduce the friction through the formation of a homogeneous water film. The patients concerned have less pain, and the risk of injury to the vessel walls is reduced as a result. Furthermore, when catheters are used, there is always a risk of blood clots forming.

Suitability for the production of such surfaces is possessed in principle by polyurethane coatings which are produced starting from solutions or dispersions of corresponding polyurethanes.

Thus U.S. Pat. No. 5,589,563 describes the use of coatings having surface-modified end groups for polymers which are used in the biomedical sector and which can also be used for the coating of medical devices. The resulting coatings are produced on the basis of solutions or dispersions, and the polymeric coatings comprise different end groups, selected from amines, fluorinated alkanols, polydimethylsiloxanes and amine-terminated polyethylene oxides. These polymers, however, do not have satisfactory properties as a coating for medical devices, more particularly in respect of the required hydrophilicity.

A disadvantage of aqueous dispersions of the kind described inter alia in U.S. Pat. No. 5,589,563 is the fact, furthermore, that the coatings are relatively rough owing to the size of the dispersed particles. Furthermore, the resulting coatings from aqueous dispersions are generally formed with inadequate stability. Consequently there is a need in respect of hydrophilic coating systems which exhibit outstanding hydrophilicity and at the same time a relatively smooth surface and a high stability. Polyurethane solutions per se are known from the prior art but have not—with the exception of the aforementioned polyurethane solutions of U.S. Pat. No. 5,589,563—been used for the coating of medical devices.

Thus, for example, DE 22 21 798 A describes a process for producing stable and light-stable solutions of polyurethaneureas from prepolymers with terminal isocyanate groups and diamines in low-polarity solvents, in which prepolymers formed from the group consisting of
a) substantially linear polyhydroxyl compounds having molecular weights of about 500 to 5000,
b) if appropriate, low molecular mass dihydroxy compounds and
c) aliphatic and/or cycloaliphatic diisocyanates, the molar ratio of hydroxyl groups and isocyanate groups being between about 1:1.5 and 1:5,
   are reacted in a solvent (mixture) comprising, if appropriate, chlorinated aromatic and/or chlorinated aliphatic hydrocarbons and primary, secondary and/or tertiary aliphatic and/or cycloaliphatic alcohols, with diamines as chain extenders, at least 80 mol % of the chain extender being 1,4-diaminocyclohexane with a cis/trans isomer ratio between 10/90 and 60/40. These polyurethaneurea solutions are used for producing lightfast films and coatings.

Furthermore, DE 22 52 280 A describes a process for the coating of textile substrates by the reverse method, with adhesion coats and topcoats comprising solutions of aliphatic, segmented polyurethane elastomers, which are polycarbonate-containing.

Furthermore, EP 0 125 466 A describes a process for the multi-coat reverse coating of textile substrates, preferably in web form, for the production of artificial leathers, made up of at least one topcoat solution and at least one adhesion coat solution on the basis of polyurethanes.

None of these publications describes a hydrophilic polyurethane resin solution which is used for coating purposes on medical devices and which meets the requirements defined above.

It is an object of the present invention, therefore, to provide a composition which is suitable for coating medical devices with hydrophilic surfaces. Since these surfaces are frequently used in blood contact, the surfaces of these materials ought also to possess good blood compatibility and ought more particularly to reduce the risk of blood clots being formed. Furthermore, the resulting coating should be smooth and have a high stability.

This invention provides coating compositions in the form of specific polyurethane solutions.

The polyurethane solutions of the invention comprise at least one polyurethaneurea which is terminated with a copolymer unit comprising polyethylene oxide and polypropylene oxide.

In accordance with the invention it has been found that compositions comprising these specific polyurethaneureas in solutions are outstandingly suitable for producing coatings on medical devices, to which they impart an outstanding hydrophilic coating, form smooth surfaces, have a high stability, and at the same time reduce the risk of blood clots forming during treatment with the medical device.

Polyurethaneureas for the purposes of the present invention are polymeric compounds which have
(a) repeat units containing at least two urethane groups, of the following general structure

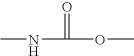

and
(b) at least one repeat unit containing urea groups

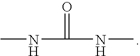

The coating compositions for use in accordance with the invention in the form of a solution are based on polyurethaneureas which have substantially no ionic modification. By this is meant, in the context of the present invention, that the polyurethaneureas for use in accordance with the invention have essentially no ionic groups, such as, more particularly, no sulphonate, carboxylate, phosphate and phosphonate groups.

The term “essentially no ionic groups” means for the purposes of the present invention that the resulting coating of the polyurethaneurea contains ionic groups with a fraction of in general not more than 2.50% by weight, more particularly not more than 2.00% by weight, preferably not more than 1.50% by weight, more preferably not more than 1.00% by weight, and especially not more than 0.50% by weight, and even more especially contains no ionic groups. Hence it is particularly preferred that the polyurethaneurea contain no ionic groups, since high concentration of ions in organic solution result in the polymer no longer being sufficiently soluble and hence in it not being possible to obtain stable solutions. If the polyurethane used in accordance with the invention contains ionic groups, then the species in question are preferably carboxylates.

The coating composition of the invention in the form of a solution comprises polyurethanes which are preferably substantially linear molecules, but may also be branched, although this is less preferred. By substantially linear molecules are meant in the context of the present invention systems with a low level of incipient crosslinking, comprising a polyol component having an average hydroxyl functionality of preferably 1.7 to 2.3, more particularly 1.8 to 2.2, more preferably 1.9 to 2.1.

The number-average molecular weight of the polyurethaneureas used with preference in accordance with the invention is preferably 1000 to 200 000, more preferably from 5000 to 100 000. The number-average molecular weight here is measured against polystyrene as standard in dimethylactamide at 30° C.

### Polyurethaneureas

The coating systems to be used according to the invention and based on polyurethaneureas are described in more detail below.

The coating compositions of the invention in the form of a solution and comprising polyurethane are prepared by reaction of synthesis components which encompass at least one polycarbonate polyol component, at least one polyisocyanate component, at least one polyoxyalkylene ether component, at least one diamine and/or amino alcohol component and, if desired, further polyol components.

The individual synthesis components are now described in more detail below.

### (a) Polycarbonate Polyol

The coating composition of the invention based on polyurethaneurea and in the form of a solution has units which originate from at least one hydroxyl-containing polycarbonate.

Suitable in principle for the introduction of units based on a hydroxyl-containing polycarbonate are polyhydroxyl compounds, having an average hydroxyl functionality of 1.7 to 2.3, preferably of 1.8 to 2.2, more preferably of 1.9 to 2.1.

Suitable hydroxyl-containing polycarbonates are polycarbonates of a molecular weight determined via the OH number of preferably 400 to 6000 g/mol, more preferably 500 to 5000 g/mol, more particularly of 600 to 3000 g/mol, which are obtainable, for example, through reaction of carbonic acid derivatives, such as diphenyl carbonate, dimethyl carbonate or phosgene, with polyols, preferably diols. Examples of suitable such diols include ethylene glycol, 1,2- and 1,3-propanediol, 1,3- and 1,4-butanediol, 1,6-hexanediol, 1,8-octanediol, neopentyl glycol, 1,4-bishydroxymethylcyclohexane, 2-methyl-1,3-propanediol, 2,2,4-trimethylpentane-1,3-diol, di-, tri- or tetraethylene glycol, dipropylene glycol, polypropylene glycols, dibutylene glycol, polybutylene glycols, bisphenol A, tetrabromobisphenol A, and also lactone-modified diols.

The diol component preferably contains 40% to 100% by weight of hexanediol, preferably 1,6-hexanediol and/or hexanediol derivatives, preferably those which as well as terminal OH groups contain ether or ester groups, examples being products obtained by reaction of 1 mol of hexanediol with at least 1 mol, preferably 1 to 2 mol, of caprolactone or through etherification of hexanediol with itself to give the di- or trihexylene glycol. Polyether-polycarbonate diols as well can be used. The hydroxyl polycarbonates ought to be substantially linear. If desired, however, they may be slightly branched as a result of the incorporation of polyfunctional components, more particularly low molecular weight polyols. Examples of those suitable for this purpose include glycerol, trimethylolpropane, hexane-1,2,6-triol, butane-1,2,4-triol, trimethylolpropane, pentaerythritol, quinitol, mannitol, sorbitol, methylglycoside or 1,3,4,6-dianhydrohexitols. Preferred polycarbonates are those based on hexane-1,6-diol, and also on co-diols with a modifying action such as butane-1,4-diol, for example, or else on ε-caprolactone. Further preferred polycarbonate diols are those based on mixtures of hexane-1,6-diol and butane-1,4-diol.

The polycarbonate is preferably substantially linear in form and includes only slight three-dimensional crosslinking, meaning that polyurethanes are formed which have the specification stated above.

### (b) Polyisocyanate

The coating composition of the invention based on polyurethaneurea and in the form of a solution has units which originate from at least one polyisocyanate as synthesis component.

As polyisocyanates (b) it is possible to use all of the aromatic, araliphatic, aliphatic and cycloaliphatic isocyanates that are known to the skilled person and have an average NCO functionality ≧1, preferably ≧2, individually or in any desired mixtures with one another, irrespective of whether they have been prepared by phosgene or phosgene-free processes. They may also contain iminooxadiazinedione, isocyanurate, uretdione, urethane, allophanate, biuret, urea, oxadiazinetrione, oxazolidinone, acylurea and/or carbodiimide structures. The polyisocyanates may be used individually or in any desired mixtures with one another.

Preference is given to using isocyanates from the series of the aliphatic or cycloaliphatic representatives, which have a carbon backbone (without the NCO groups present) of 3 to 30, preferably 4 to 20, carbon atoms.

Particularly preferred compounds of component (b) conform to the type specified above having aliphatically and/or cycloaliphatically attached NCO groups, such as, for example, bis(isocyanatoalkyl)ethers, bis- and tris(isocyanatoalkyl)benzenes, -toluenes, and -xylenes, propane diisoscyanates, butane diisocyanates, pentane diisocyanates, hexane diisocyanates (e.g. hexamethylene diisocyanate, HDI), heptane diisocyanates, octane diisocyanates, nonane diisocyanates (e.g. trimethyl-HDI (TMDI), generally as a mixture of the 2,4,4 and 2,2,4 isomers), nonane triisocyanates (e.g. 4-isocyanatomethyl-1,8-octane diisocyanate), decane diisocyanates, decane triisocyanates, undecane diisocyanates, undecane triisocyanates, dodecane diisocyanates, dodecane triisocyanates, 1,3- and 1,4-bis(isocyanatomethyl)cyclohexanes (H₆XDI), 3-isocyanatomethyl-3,5,5-trimethylcyclohexyl isocyanate (isophorone diisocyanate, IPDI), bis(4-isocyanatocyclohexyl)methane (H₁₂MDI) or bis(isocyanatomethyl)norbornane (NBDI).

Very particularly preferred compounds of component (b) are hexamethylene diisocyanate (HDI), trimethyl-HDI (TMDI), 2-methylpentane 1,5-diisocyanate (MPDI), isophorone diisocyanate (IPDI), 1,3- and 1,4-bis(isocyanatomethyl)cyclohexane (H₆XDI), bis(isocyanatomethyl)norbornane (NBDI), 3(4)-isocyanatomethyl-1-methylcyclohexyl isocyanate (IMCI) and/or 4,4′-bis(isocyanatocyclohexyl)methane (H₁₂MDI) or mixtures of these isocyanates. Further examples are derivatives of the above diisocyanates with a uretdione, isocyanurate, urethane, allophanate, biuret, iminooxadiazinedione and/or oxadiazinetrione structure and with more than two NCO groups.

The amount of constituent (b) in the coating composition for use in accordance with the invention is preferably 1.0 to 3.5 mol, more preferably 1.0 to 3.3 mol, more particularly 1.0 to 3.0 mol, based in each case on the constituent (a) of the coating composition for use in accordance with the invention.

### (c) Polyoxyalkylene Ethers

The polyurethaneurea used in the present invention has units which originate from a copolymer comprising polyethylene oxide and polypropylene oxide as synthesis component. These copolymer units are present in the form of end groups in the polyurethaneurea and bring about the hydrophilicization of the coating composition of the invention.

Nonionically hydrophilicizing compounds (c) are, for example, monofunctional polyalkylene oxide polyether alcohols containing an average 5 to 70, preferably 7 to 55, ethylene oxide units per molecule, of the kind available in conventional manner through alkoxylation of suitable starter molecules (e.g. in Ullmanns Enzyklopädie der technischen Chemie, 4th Edition, Volume 19, Verlag Chemie, Weinheim pp. 31-38).

Examples of suitable starter molecules are saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, such as diethylene glycol monobutyl ether, for example, unsaturated alcohols such as allyl alcohol, 1,1-dimethylallyl alcohol or oleyl alcohol, aromatic alcohols such as phenol, the isomeric cresols or methoxyphenols, araliphatic alcohols such as benzyl alcohol, anisyl alcohol or cinnamyl alcohol, secondary monoamines such as dimethylamine, diethylamine, dipropylamine, diisopropylamine, dibutylamine, bis(2-ethylhexyl)amine, N-methyl- and N-ethylcyclohexylamine or dicyclohexylamine, and also heterocyclic secondary amines such as morpholine, pyrrolidine, piperidine or 1H-pyrazole. Preferred starter molecules are saturated monoalcohols. Particular preference is given to using diethylene glycol monobutyl ether as a starter molecule.

The alkylene oxides, ethylene oxide and propylene oxide, can be used in any order or else in a mixture in the alkoxylation reaction.

Polyalkylene oxide polyether alcohols are mixed polyalkylene oxide polyethers of ethylene oxide and propylene oxide, whose alkylene oxide units are composed preferably to an extent of at least 30 mol %, more preferably at least 40 mol %, of ethylene oxide units. Preferred non-ionic compounds are monofunctional mixed polyalkylene oxide polyethers which contain at least 40 mol % of ethylene oxide units and not more than 60 mol % of propylene oxide units.

The average molar weight of the polyoxyalkylene ether is preferably 500 g/mol to 5000 g/mol, more preferably 1000 g/mol to 4000 g/mol, more preferably 1000 to 3000 g/mol.

The amount of constituent (c) in the coating composition for use in accordance with the invention is preferably 0.01 to 0.5 mol, more preferably 0.02 to 0.4 mol, more particularly 0.04 to 0.3 mol, based in each case on constituent (a) of the coating composition for use in accordance with the invention.

In accordance with the invention it has been possible to show that the polyurethaneureas with end groups based on mixed polyoxyalkylene ethers comprising polyethylene oxide and polypropylene oxide are especially suitable for producing coatings having a high hydrophilicity. As will be shown later on below, in comparison to polyurethaneureas terminated only by polyethylene oxide, the coatings of the invention effect a significantly low contact angle and are therefore more hydrophilic in form.

### (d) Diamine or Amino Alcohol

The polyurethaneurea solution of the invention has units which originate from at least one diamine or amino alcohol as synthesis components and serve as what are called chain extenders (d).

Examples of such chain extenders are diamines or polyamines and also hydrazides, e.g. hydrazine, ethylenediamine, 1,2- and 1,3-diaminopropane, 1,4-diaminobutane, 1,6-diaminohexane, isophoronediamine, isomer mixture of 2,2,4- and 2,4,4-trimethylhexamethylenediamine, 2-methylpentamethylenediamine, diethylenetriamine, 1,3- and 1,4-xylylenediamine, α,α,α′,α′-tetramethyl-1,3- and -1,4-xylylenediamine and 4,4′-diaminodicyclohexylmethane, dimethylethylenediamine, hydrazine, adipic dihydrazide, 1,4-bis(aminomethyl)cyclohexane, 4,4′-diamino-3,3′-dimethyldicyclohexylmethane and other (C₁-C₄) di- and tetraalkyldicyclohexylmethanes, e.g. 4,4′-diamino-3,5-diethyl-3′,5′-diisopropyldicyclohexylmethane.

Suitable diamines or amino alcohols are generally low molecular weight diamines or amino alcohols which contain active hydrogen with differing reactivity towards NCO groups, such as compounds which as well as a primary amino group also contain secondary amino groups or which as well as amino group (primary or secondary) also contain OH groups. Examples of such compounds are primary and secondary amines, such as 3-amino-1-methylaminopropane, 3-amino-1-ethylaminopropane, 3-amino-1-cyclohexylaminopropane, 3-amino-1-methylaminobutane, and also amino alcohols, such as N-aminoethylethanolamine, ethanolamine, 3-aminopropanol, neopentanolamine and, with particular preference, diethanolamine.

The constituent (d) of the coating composition for use in accordance with the invention can be used, in the context of the preparation of the composition, as a chain extender.

The amount of constituent (d) in the coating composition solution of the invention is preferably 0.1 to 1.5 mol, more preferably 0.2 to 1.3 mol, more particularly 0.3 to 1.2 mol, based in each case on constituent (a) of the coating composition for use in accordance with the invention.

### (e) Polyols

In a further embodiment the coating composition of the invention in the form of a solution comprises additional units which originate from at least one further polyol as synthesis component.

The further low molecular weight polyols (e) used to synthesize the polyurethaneureas have the effect, generally, of stiffening and/or branching the polymer chain. The molecular weight is preferably 62 to 500 g/mol, more preferably 62 to 400 g/mol, more particularly 62 to 200 g/mol.

Suitable polyols may contain aliphatic, alicyclic or aromatic groups. Mention may be made here, for example, of the low molecular weight polyols having up to about 20 carbon atoms per molecule, such as, for example, ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxyphenyl)propane), hydrated bisphenol A (2,2-bis(4-hydroxycyclohexyl)propane), and also trimethylolpropane, glycerol or pentaerythritol, and mixtures of these and, if desired, other low molecular weight polyols as well. Use may also be made of ester diols such as, for example, α-hydroxybutyl-ε-hydroxycaproic acid ester, ω-hydroxyhexyl-γ-hydroxybutyric acid ester, adipic acid (β-hydroxyethyl) ester or terephthalic acid bis(β-hydroxyethyl) ester.

The amount of constituent (e) in the coating composition for use in accordance with the invention is preferably 0.05 to 1.0 mol, more preferably 0.05 to 0.5 mol, more particularly 0.1 to 0.5 mol, based in each case on constituent (a) of the coating composition for use in accordance with the invention.

(f) Further Amine- and/or Hydroxy-Containing Units (Synthesis Component)

The reaction of the isocyanate-containing component (b) with the hydroxy- or amine-functional compounds (a), (c), (d) and, if used, (e) takes place typically with a slight NCO excess being observed over the reactive hydroxy or amine compounds. At the endpoint of the reaction, through achievement of a target viscosity, there always still remain residues of active isocyanate. These residues must be blocked, so that there is no reaction with large polymer chains. Such a reaction leads to the three-dimensional crosslinking and gelling of the batch. The processing of such a coating solution is no longer possible. Customarily the batches include high quantities of alcohols. Within a number of hours of standing or stirring of the batch, at room temperature, these alcohols block the isocyanate groups that still remain.

If, however, it is desired to carry out rapid blocking of the residual isocyanate content that still remains, the polyurethaneurea coating compositions provided in accordance with the invention in the form of a solution may also include monomers (f) as synthesis components, which are in each case located at the chain ends and cap them.

These synthesis components derive on the one hand from monofunctional compounds that are reactive with NCO groups, such as monoamines, more particularly mono-secondary amines, or monoalcohols. Mention may be made here, for example, of ethanol, n-butanol, ethylene glycol monobutyl ether, 2-ethylhexanol, 1-octanol, 1-dodecanol, 1-hexadecanol, methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine and suitable substituted derivatives thereof.

Since the units (f) are used essentially in the coating composition of the invention in the form of a solution to destroy the NCO excess, the amount required is dependent essentially on the amount of the NCO excess, and cannot be specified generally.

Preferably these units are not used during the synthesis. Unreacted isocyanate is preferably converted into terminal urethanes as a result of the solvent alcohols that are present at very high concentrations.

### (g) Further Constituents

Furthermore, the solutions of the polyurethaneurea coating composition provided according to the invention may comprise further constituents and additives typical for the intended purpose. An example of such are active pharmacological substances, medicaments, and additives which promote the release of active pharmacological substances (drug-eluting additives).

Pharmacological substances or medicaments which may be used in the coatings of the invention on the medical devices and can thus be contained in the solutions of the invention are, for example, thromboresistant agents, antibiotic agents, antitumour agents, growth hormones, antiviral agents, antiangiogenic agents, angiogenic agents, antimitotic agents, anti-inflammatory agents, cell cycle regulators, genetic agents, hormones, and also their homologues, derivatives, fragments, pharmaceutical salts, and combinations thereof.

Specific examples of such pharmacological substances or medicaments hence include thromboresistant (non-thrombogenic) agents and other agents for suppressing acute thrombosis, stenosis or late restenosis of the arteries, examples being heparin, streptokinase, urokinase, tissue plasminogen activator, anti-thromboxan-B₂ agent; anti-B-thromboglobulin, prostaglandin-E, aspirin, dipyridimol, anti-thromboxan-A₂ agent, murine monoclonal antibody 7E3, triazolopyrimidine, ciprostene, hirudin, ticlopidine, nicorandil, etc. A growth factor can likewise be utilized as a medicament in order to suppress subintimal fibromuscular hyperplasia at the arterial stenosis site, or any other cell growth inhibitor can be utilized at the stenosis site.

The pharmacological substance or medicament may also be composed of a vasodilatator, in order to counteract vaso-spasm—for example, an antispasm agent such as papaverine. The medicament may be a vasoactive agent per se, such as calcium antagonists, or α- and β-adrenergic agonists or antagonists. In addition the therapeutic agent may be a biological adhesive such as cyanoacrylate in medical grade, or fibrin, which is used, for example, for bonding a tissue valve to the wall of a coronary artery.

The therapeutic agent may further be an antineoplastic agent such as 5-fluorouracil, preferably with a controlling releasing vehicle for the agent (for example, for the use of an ongoing controlled releasing antineoplastic agent at a tumour site).

The therapeutic agent may be an antibiotic, preferably in combination with a controlling releasing vehicle for the ongoing release from the coating of a medical device at a localized focus of infection within the body. Similarly, the therapeutic agent may comprise steroids for the purpose of suppressing inflammation in localized tissue, or for other reasons.

Specific examples of suitable medicaments include:
(a) heparin, heparin sulphate, hirudin, hyaluroic acid, chondroitin sulphate, dermatan sulphate, keratan sulphate, lytic agents, including urokinase and streptokinase, their homologues, analogues, fragments, derivatives and pharmaceutical salts thereof;
(b) antibiotic agents such as penicillins, cephalosporins, vacomycins, aminoglycosides, quinolones, polymyxins, erythromycins; tetracyclines, chloramphenicols, clindamycins, lincomycins, sulphonamides, their homologues, analogues, derivatives, pharmaceutical salts and mixtures thereof;
(c) paclitaxel, docetaxel, immunosuppressants such as sirolimus or everolimus, alkylating agents, including mechlorethamine, chlorambucil, cyclophosphamide, melphalane and ifosfamide; antimetabolites, including methotrexate, 6-mercaptopurine, 5-fluorouracil and cytarabine; plant alkaloids, including vinblastin; vincristin and etoposide; antibiotics, including doxorubicin, daunomycin, bleomycin and mitomycin; nitrosurea, including carmustine and lomustine; inorganic ions, including cisplatin; biological reaction modifiers, including interferon; angiostatins and endostatins; enzymes, including asparaginase; and hormones, including tamoxifen and flutamide, their homologues, analogues, fragments, derivatives, pharmaceutical salts and mixtures thereof;
(d) antiviral agents such as amantadine, rimantadine, rabavirin, idoxuridine, vidarabin, trifluridine, aciclovir, ganciclovir, zidovudine, phosphonoformates, interferons, their homologues, analogues, fragments, derivatives, pharmaceutical salts and mixtures thereof; and
e) antiinflammatory agents such as, for example, ibuprofen, dexamethasone or methylprednisolone.

In one preferred embodiment the coating composition of the invention in the form of a solution comprises a polyurethaneurea which is at least synthesized from
a) at least one polycarbonate polyol;
b) at least one polyisocyanate;
c) at least one monofunctional polyoxyalkylene ether; and
d) at least one diamine or amino alcohol.

In a further preferred embodiment of the present invention the coating composition of the invention in the form of a solution comprises a polyurethaneurea which is at least synthesized from
a) at least one polycarbonate polyol;
b) at least one polyisocyanate;
c) at least one monofunctional polyoxyalkylene ether;
d) at least one diamine or amino alcohol; and
e) at least one polyol.

In a further preferred embodiment of the present invention the coating composition of the invention in the form of a solution comprises a polyurethaneurea which is at least synthesized from
a) at least one polycarbonate polyol;
b) at least one polyisocyanate;
c) at least one monofunctional polyoxyalkylene ether;
d) at least one diamine or amino alcohol;
e) at least one polyol; and
f) at least one further amine- and/or hydroxy-containing unit.

The coating compositions of the invention in the form of a solution preferably comprise polyurethaneureas which are at least synthesized from
a) at least one polycarbonate polyol having an average molar weight between 400 g/mol and 6000 g/mol and a hydroxyl functionality of 1.7 to 2.3, or mixtures of such polycarbonate polyols;
b) at least one aliphatic, cycloaliphatic or aromatic polyisocyanate or mixtures of such polyisocyanates in an amount per mole of the polycarbonate polyol of 1.0 to 3.5 mol;
c) at least one monofunctional polyoxyalkylene ether or a mixture of such polyethers, having an average molar weight between 500 g/mol and 5000 g/mol, in an amount per mole of the polycarbonate polyol of 0.01 to 0.5 mol;
d) at least one aliphatic or cycloaliphatic diamine or at least one amino alcohol, as so-called chain extenders, or mixtures of such compounds in an amount per mole of the polycarbonate polyol of 0.1 to 1.5 mol;
e) if desired, one or more short-chain aliphatic polyols having a molar weight between 62 g/mol and 500 g/mol, in an amount per mole of the polycarbonate polyol of 0.05 to 1 mol; and
f) if desired, amine- or OH-containing units which are located on, and cap, the polymer chain ends.

Preference is further given in accordance with the invention to using polyurethaneureas in the coating composition in the form of a solution which are at least synthesized from
a) at least one polycarbonate polyol having an average molar weight between 500 g/mol and 5000 g/mol and a hydroxyl functionality of 1.8 to 2.2, or mixtures of such polycarbonate polyols;
b) at least one aliphatic, cycloaliphatic or aromatic polyisocyanate or mixtures of such polyisocyanates in an amount per mole of the polycarbonate polyol of 1.0 to 3.3 mol;
c) at least one monofunctional polyoxyalkylene ether or a mixture of such polyethers, having an average molar weight between 1000 g/mol and 4000 g/mol, in an amount per mole of the polycarbonate polyol of 0.02 to 0.4 mol;
d) at least one aliphatic or cycloaliphatic diamine or at least one amino alcohol, as so-called chain extenders, or mixtures of such compounds in an amount per mole of the polycarbonate polyol of 0.2 to 1.3 mol;
e) if desired, one or more short-chain aliphatic polyols having a molar weight between 62 g/mol and 400 g/mol, in an amount per mole of the polycarbonate polyol of 0.05 to 0.5 mol; and
f) if desired, amine- or OH-containing units which are located on, and cap, the polymer chain ends.

Preference is also further given in accordance with the invention to using polyurethaneureas in the coating solution which are at least synthesized from
a) at least one polycarbonate polyol having an average molar weight between 600 g/mol and 3000 g/mol and a hydroxyl functionality of 1.9 to 2.1, or mixtures of such polycarbonate polyols;
b) at least one aliphatic, cycloaliphatic or aromatic polyisocyanate or mixtures of such polyisocyanates in an amount per mole of the polycarbonate polyol of 1.0 to 3.0 mol;
c) at least one monofunctional polyoxyalkylene ether or a mixture of such polyethers, having an average molar weight between 1000 g/mol and 3000 g/mol, in an amount per mole of the polycarbonate polyol of 0.04 to 0.3 mol, more particular preference being given to a mixture of polyethylene oxide and polypropylene oxide; and
d) at least one aliphatic or cycloaliphatic diamine or at least one amino alcohol, as so-called chain extenders, or mixtures of such compounds in an amount per mole of the polycarbonate polyol of 0.3 to 1.2 mol; and
e) if desired, one or more short-chain aliphatic polyols having a molar weight between 62 g/mol and 400 g/mol, in an amount per mole of the polycarbonate polyol of 0.1 to 0.5 mol.

### Use of the Inventive Coating Composition in the Form of a Solution

The coating composition of the invention in the form of a solution can be used to form a coating on a medical device.

The term “medical device” is to be understood broadly in the context of the present invention. Suitable, non-limiting examples of medical devices (including instruments) are contact lenses; cannulas; catheters, for example urology catheters such as urinary catheters or ureteral catheters; central venous catheters; venous catheters or inlet or outlet catheters; dilation balloons; catheters for angioplasty and biopsy; catheters used for introducing a stent, an embolism filter or a vena caval filter; balloon catheters or other expandable medical devices; endoscopes; laryngoscopes; tracheal devices such as endotracheal tubes, respirators and other tracheal aspiration devices; bronchoalveolar lavage catheters; catheters used in coronary angioplasty; guide rods, insertion guides and the like; vascular plugs; pacemaker components; cochlear implants; dental implant tubes for feeding, drainage tubes; and guide wires.

The coating solutions of the invention may be used, furthermore, for producing protective coatings, for example for gloves, stents and other implants; external (extracorporeal) blood lines (blood-carrying pipes); membranes, for example for dialysis; blood filters; devices for circulatory support; dressing material for wound management; urine bags and stoma bags. Also included are implants which comprise a medically active agent, such as medically active agents for stents or for balloon surfaces or for contraceptives.

Typically the medical device is formed from catheters, endoscopes, laryngoscopes, endotracheal tubes, feeding tubes, guide rods, stents, and other implants.

There are many materials suitable as a substrate of the surface to be coated, such as metals, textiles, ceramics or plastics, the use of plastics being preferred for the production of medical devices.

In accordance with the invention it has been found that it is possible to produce medical devices having very hydrophilic and hence lubricous, blood-compatible surfaces by using aqueous, nonionically stabilized polyurethane dispersions of the type described above to coat the medical devices. The coating compositions described above are obtained preferably as organic solution and are applied to the surface of the medical devices.

Preference is given in this context, more particularly, to coating solutions synthesized from a mixture of polycarbonate polyols and a monofunctional polypropylene oxide-polyethylene oxide alcohol.

### Preparation of the Coating Solutions

In the context of the present invention it is more particularly preferable that the coatings on the medical devices are produced starting from solutions of the coating composition described in more detail above.

In accordance with the invention it has emerged that the resulting coatings on medical devices differ according to whether the coating composition described above is prepared starting from a dispersion or from a solution.

In this context the coatings of the invention on medical devices have advantages when they are obtained starting from solutions of the above-described coating compositions.

Without wishing to be tied to any one theory, it is nevertheless assumed in accordance with the invention that by virtue of the particulate structure of the polyurethanes in aqueous dispersion the filming of the polymers is incomplete. In the films, the particle structures can generally still be seen, as for example by atomic force microscopy (AFM). Polyurethanes from solution yield smoother coatings. As a result of the internal interlocking and interlooping of the polyurethane molecules in organic solution, the dried films as well have greater tensile strength and are more resistant to storage in water.

The medical devices of the invention can be coated with the hydrophilic polyurethane solutions by means of a variety of methods. Examples of suitable coating techniques for this purpose include knifecoating, printing, transfer coating, spraying, spin coating or dipping.

The organic polyurethane solutions can be prepared by any desired methods.

A procedure which has been found to be preferred, however, is as follows:

For the preparation of the polyurethaneurea solutions for use in accordance with the invention for coating, preferably the polycarbonate polyol, the polyisocyanate, the monofunctional polyether alcohol and, if desired, the polyol are reacted with one another in the melt or in solution, until all of the hydroxyl groups have been consumed.

The stoichiometry used in this case between the individual synthesis components involved in the reaction is a product of the aforementioned proportions.

The reaction takes place at a temperature of preferably between 60 and 110° C., more preferably 75 to 110° C., more particularly 90 to 110° C., with temperatures around 110° C. being preferred on account of the rate of the reaction. Higher temperatures may likewise be employed, although in that case there is a risk, in certain cases and as a function of the individual constituents used, that decomposition processes and instances of discoloration will appear in the resultant polymer.

In the case of the prepolymer of isocyanate and all hydroxyl-containing components, reaction in the melt is preferred, although there is a risk of high viscosities in the fully reacted mixtures. In these cases it is also advisable to add solvents. However, as far as possible there should not be more than about 50% by weight of solvent present, since otherwise the dilution makes the reaction rate significantly slower.

In the case of the reaction of isocyanate and the hydroxyl-containing components, the reaction may take place in the melt in a period of 1 hour to 24 hours. Low levels of addition of quantities of solvent lead to a slowing down, but the reaction times are within the same periods.

The sequence of the addition or reaction of the individual constituents may differ from the sequence indicated above. This may be of advantage more particularly when the intention is that the mechanical properties of the resultant coatings should be changed. If, for example, all of the hydroxyl-containing components are reacted at the same time, the product is a mixture of hard and soft segments. If, for example, the low molecular mass polyol is added after the polycarbonate polyol component, defined blocks are obtained, and this may be accompanied by different properties in the resultant coatings. The present invention is hence not restricted to an arbitrary sequence of the addition or reaction of the individual constituents of the polyurethane coating.

Then further solvent is added and the chain extender diamine, in solution if appropriate, or the dissolved chain extender amino alcohol (synthesis component (d)) is added.

The further addition of the solvent takes place preferably in steps, so as not to slow down the reaction unnecessarily, which would occur in the case of complete addition of the quantity of solvent at the beginning of the reaction, for example. Furthermore, in the case of a high level of solvent at the beginning of the reaction, one is tied to a relatively low temperature, which is at least co-determined by the nature of the solvent. This too leads to a slowing down of the reaction.

When the target viscosity has been reached, the residues of NCO that still remain can be blocked by means of a monofunctional aliphatic amine. The remaining isocyanate groups are preferably blocked by reaction with the alcohols that are present in the solvent mixture.

Suitable solvents for the preparation and the application of the polyurethaneurea solutions of the invention include all conceivable solvents and solvent mixtures such as dimethylformamide, N-methylacetamide, tetramethylurea, N-methylpyrrolidone, aromatic solvents such as toluene, linear and cyclic esters, ethers, ketones and alcohols. Examples of esters and ketones are, for example, ethyl acetate, butyl acetate, acetone, γ-butyrolactone, methyl ethyl ketone and methyl isobutyl ketone.

Preference is given to mixtures of alcohols with toluene. Examples of the alcohols which are used together with the toluene are ethanol, n-propanol, isopropanol and 1-methoxy-2-propanol.

Generally speaking, in the reaction, the amount of solvent used is such as to give approximately 10% to 50% strength by weight solutions, more preferably approximately 15% to 45% strength by weight solutions, with particular preference approximately 20% to 40% strength by weight solutions.

The solids content of the polyurethane solutions is generally between 5% to 60% by weight, preferably 10% to 40% by weight. For coating experiments the polyurethane solutions can be diluted as desired with toluene/alcohol mixtures in order to allow the thickness of the coating to be varied. All concentrations from 1% to 60% by weight are possible, with preferred concentrations being in the 1% to 40% by weight range.

In this context it is possible to achieve any desired coat thicknesses, such as, for example, a few 100 nm up to a few 100 μm, although higher and lower thicknesses are also possible in the context of the present invention.

Further additions, such as antioxidants or pigments, for example, can likewise be used. It is also possible if desired, furthermore, to use further additions such as hand assistants, dyes, matting agents, UV stabilizers, light stabilizers, hydrophobicizing agents, hydrophilicizing agents and/or flow control assistants.

Starting from these solutions, then, medical coatings are produced by the processes described above.

A wide variety of substrates can be coated, such as metals, textiles, ceramics and plastics.

Preference is given to coating medical devices manufactured from metals or from plastic. Examples of metals include the following: medical stainless steel or nickel titanium alloys. Many polymer materials are conceivable from which the medical device may be constructed, examples being polyamide; polystyrene; polycarbonate; polyethers; polyesters; polyvinyl acetate; natural and synthetic rubbers; block copolymers of styrene and unsaturated compounds such as ethylene, butylene and isoprene; polyethylene or copolymers of polyethylene and polypropylene; silicone; polyvinyl chloride (PVC) and polyurethanes. For better adhesion of the hydrophilic polyurethanes to the medical device, further suitable coatings may be applied as a base before these hydrophilic coating materials are applied.

The medical devices can be coated with the hydrophilic polyurethane dispersions by a variety of methods. Suitable coating techniques are knifecoating, printing, transfer coating, spraying, spin coating or dipping.

In addition to the hydrophilic properties of the improvement of slip, the coating compositions provided in accordance with the invention are also distinguished by a high level of blood compatibility. As a result, working with these coatings is also advantageous, particularly in blood contact. In comparison to polymers of the prior art, the materials exhibit reduced coagulation tendency in blood contact.

The advantages of the catheters of the invention with the hydrophilic polyurethane coatings are set out by means of comparative experiments in the following examples.

### EXAMPLES

The NCO content of the resins described in the inventive and comparative examples was determined by titration in accordance with DIN EN ISO 11909.

The solids contents were determined in accordance with DIN-EN ISO 3251.1 g of polyurethane dispersion was dried at 115° C. to constant weight (15-20 min) using an infrared dryer.

The average particle sizes of the polyurethane dispersions are measured using the High Performance Particle Sizer (HPPS 3.3) from Malvern Instruments.

Unless noted otherwise, amounts indicated in % are % by weight and relate to the aqueous dispersion obtained.

Viscosity measurements were carried out using the Physics MCR 51 rheometer from Anton Paar GmbH, Ostfildern, Germany.

### Substances and Abbreviations Used:

Desmophen C2200: Polycarbonate polyol, OH number 56 mg KOH/g, number-average molecular weight 2000 g/mol (Bayer, MaterialScience AG, Leverkusen, DE)
Desmophen C1200: Polycarbonate polyol, OH number 56 mg KOH/g, number-average molecular weight 2000 g/mol (Bayer MaterialScience AG, Leverkusen, DE)
Desmophen XP 2613 Polycarbonate polyol, OH number 56 mg KOH/g, number-average molecular weight 2000 g/mol (Bayer MaterialScience AG, Leverkusen, DE)
PolyTHF® 2000: Polytetramethylene glycol polyol, OH number 56 mg KOH/g, number-average molecular weight 2000 g/mol (BASF AG, Ludwigshafen, DE)
Polyether LB 25: (mono-functional polyether based on ethylene oxide/propylene oxide, number-average molecular weight 2250 g/mol, OH number 25 mg KOH/g (Bayer MaterialScience AG, Leverkusen, DE)

### Example 1

This example describes the preparation of an inventive polyurethaneurea solution

198.6 g of Desmophen C 2200, 23.0 g of LB 25 and 47.8 g of 4,4′-bis(isocyanatocyclohexyl)methane (H₁₂MDI) were reacted at 110° C. in the melt to a constant NCO content of 2.4%. The product was cooled and diluted with 350.0 g of toluene and 200 g of isopropanol. At room temperature a solution of 12.5 g of isophoronediamine in 95.0 g of 1-methoxypropan-2-ol was added. After the end of the increase in molar weight and the attainment of the desired viscosity range (testing by measurement of the viscosity of a sample taken, using the rheometer cited above), the batch was stirred for a further 4 hours in order to block the remaining isocyanate content with isopropanol. This gave 927 g of a 30.4% strength solution of polyurethaneurea in toluene/isopropanol/1-methoxypropan-2-ol with a viscosity of 19 600 mPas at 22° C.

### Example 2

This example describes the preparation of an inventive polyurethaneurea solution

195.4 g of Desmophen C 2200, 30.0 g of LB 25 and 47.8 g of 4,4′-bis(isocyanatocyclohexyl)methane (H₁₂MDI) were reacted at 110° C. to a constant NCO content of 2.3%. The product was cooled and diluted with 350.0 g of toluene and 200 g of isopropanol. At room temperature a solution of 12.7 g of isophoronediamine in 94.0 g of 1-methoxypropan-2-ol was added. After the end of the increase in molar weight and the attainment of the desired viscosity range, the batch was stirred for a further 4 hours in order to block the remaining isocyanate content with isopropanol. This gave 930 g of a 30.7% strength solution of polyurethaneurea in toluene/isopropanol/1-methoxypropan-2-ol with a viscosity of 38 600 mPas at 22° C.

### Example 3

This example describes the preparation of an inventive polyurethaneurea solution

195.4 g of Desmophen XP 2613, 30.0 g of LB 25 and 47.8 g of 4,4′-bis(isocyanatocyclohexyl)methane (H₁₂MDI) were reacted at 110° C. to a constant NCO content of 2.4%. The product was cooled and diluted with 350.0 g of toluene and 200 g of isopropanol. At room temperature a solution of 12.7 g of isophoronediamine in 95.0 g of 1-methoxypropan-2-ol was added. After the end of the increase in molar weight and the attainment of the desired viscosity range, the batch was stirred for a further 4 hours in order to block the remaining isocyanate content with isopropanol. This gave 931 g of a 30.7% strength solution of polyurethaneurea in toluene/isopropanol/1-methoxypropan-2-ol with a viscosity of 26 500 mPas at 22° C.

### Example 4

This example describes the preparation of an inventive polyurethaneurea solution

198.6 g of Desmophen C 1200, 23.0 g of LB 25 and 47.8 g of 4,4′-bis(isocyanatocyclohexyl)methane (H₁₂MDI) were reacted at 110° C. to a constant NCO content of 2.4%. The product was cooled and diluted with 350.0 g of toluene and 200 g of isopropanol. At room temperature a solution of 13.2 g of isophoronediamine in 100.0 g of 1-methoxypropan-2-ol was added. After the end of the increase in molar weight and the attainment of the desired viscosity range, the batch was stirred for a further 4 hours in order to block the remaining isocyanate content with isopropanol. This gave 933 g of a 30.3% strength solution of polyurethaneurea in toluene/isopropanol/1-methoxypropan-2-ol with a viscosity of 17 800 mPas at 22° C.

### Example 5

This example describes the preparation of an inventive polyurethaneurea solution

195.4 g of Desmophen C 1200, 30.0 g of LB 25 and 47.8 g of 4,4′-bis(isocyanatocyclohexyl)methane (H₁₂MDI) were reacted at 110° C. to a constant NCO content of 2.4%. The product was cooled and diluted with 350.0 g of toluene and 200 g of isopropanol. At room temperature a solution of 11.8 g of isophoronediamine in 94.0 g of 1-methoxypropan-2-ol was added. After the end of the increase in molar weight and the attainment of the desired viscosity range, the batch was stirred for a further 4 hours in order to block the remaining isocyanate content with isopropanol. This gave 931 g of a 30.7% strength solution of polyurethaneurea in toluene/isopropanol/1-methoxypropan-2-ol with a viscosity of 23 700 mPas at 22° C.

### Example 6

This example describes the preparation of a polyurethaneurea solution as a comparison product to the inventive Example 1. The Desmophen C2200 is replaced by the PolyTHF 2000.

194.0 g of PolyTHF 2000, 22.6 g of LB 25 and 47.8 g of 4,4′-bis(isocyanatocyclohexyl)methane (H₁₂MDI) were reacted at 110° C. to a constant NCO content of 2.3%. The product was cooled and diluted with 350.0 g of toluene and 200 g of isopropanol. At room temperature a solution of 12.1 g of isophoronediamine in 89.0 g of 1-methoxypropan-2-ol was added. After the end of the increase in molar weight and the attainment of the desired viscosity range, the batch was stirred for a further 4 hours in order to block the remaining isocyanate content with isopropanol. This gave 916 g of a 30.2% strength solution of polyurethaneurea in toluene/isopropanol/1-methoxypropan-2-ol with a viscosity of 15 200 mPas at 22° C.

### Example 7

This example describes the preparation of a polyurethaneurea solution as a comparison product to the inventive Example 2. The Desmophen C2200 is replaced by the PolyTHF 2000.

190.6 g of PolyTHF 2000, 30.0 g of LB 25 and 47.8 g of 4,4′-bis(isocyanatocyclohexyl)methane (H₁₂MDI) were reacted at 110° C. to a constant NCO content of 2.3%. The product was cooled and diluted with 350.0 g of toluene and 200 g of isopropanol. At room temperature a solution of 12.1 g of isophoronediamine in 89.0 g of 1-methoxypropan-2-ol was added. After the end of the increase in molar weight and the attainment of the desired viscosity range, the batch was stirred for a further 4 hours in order to block the remaining isocyanate content with isopropanol. This gave 919 g of a 30.5% strength solution of polyurethaneurea in toluene/isopropanol/1-methoxypropan-2-ol with a viscosity of 21 000 mPas at 22° C.

### Example 8

### Production of the Coatings and Measurement of the Static Contact Angle

The coatings for the measurement of the static contact angle were produced on glass slides measuring 25×75 mm using a spin coater (RC5 Gyrset 5, Karl Suss, Garching, Germany). For this purpose a slide was clamped onto the sample plate of the spincoater and covered homogeneously with about 2.5-3 g of organic 15% strength polyurethane solution. All of the organic polyurethane solutions were diluted to a polymer content of 15% by weight with a solvent mixture composed of 65% by weight of toluene and 35% by weight of isopropanol. Rotation of the sample plate at 1300 revolutions per minute for 20 sec gave a homogeneous coating, which was dried at 100° C. for 1 h and then at 50° C. for 24 h. The coated slides obtained were subjected directly to a contact angle measurement.

A static contact angle measurement was performed on the resulting coatings on the slides. Using the video contact angle measuring instrument OCA20 from Dataphysics, with computer-controlled injection, 10 drops of Millipore water are placed on the specimen, and their static wetting angle is measured. Beforehand, using an antistatic dryer, the static charge (if present) on the sample surface was removed.

TABLE 1
Static contact angle measurements

| PU FILM | Contact angle [°] |
|---|---|
| Inventive Example 1 | 32 |
| Inventive Example 2 | 21 |
| Inventive Example 3 | 38 |
| Inventive Example 4 | 25 |
| Inventive Example 5 | 25 |
| Example 6 (comparative example) | 83 |
| Example 7 (comparative example) | 82 |

As Table 1 shows, the polycarbonate-containing coatings of Inventive Examples 1-5 give extremely hydrophilic coatings with static contact angles ≦40°. In contrast, the PolyTHF-containing coatings 7-9 are substantially less polar, despite the fact that the composition of these coatings is otherwise identical with those of Examples 1 and 2.

### Example 9

This comparative example describes the synthesis of a polyurethaneurea polymer which in place of the mixed monofunctional polyethylene-polypropylene oxide alcohol LB 25 contains the same molar fraction of a pure monofunctional polyethylene oxide alcohol. The polymer is identical with that of Example 1 except that it contains a different terminal group. The synthesis in toluene and alcohols as described in Examples 1-7 does not function when this alcohol is used. Consequently the synthesis is carried out in pure dimethylformamide (DMF).

198.6 g of Desmophen C 2200, 20.4 g of polyethylene glycol 2000 monomethyl ether (source: Fluka, Article No. 81321) and 47.8 g of 4,4′-bis(isocyanatocyclohexyl)methane (H₁₂MDI) were reacted at 110° C. in the melt to a constant NCO content of 2.4%. The product was cooled and diluted with 550 g of DMF. At room temperature a solution of 10.5 g of isophoronediamine in 100 g of DMF was added. After the end of the increase in molar weight and the attainment of the desired viscosity range (testing by measurement of the viscosity of a sample taken, using the rheometer cited above) 1.0 g of n-butylamine was added in order to block the small isocyanate content that remained. This gave 927 g of a 29.8% strength solution of polyurethaneurea in dimethylformamide with a viscosity of 22 700 mPas at 23° C.

### Example 10

This example describes the synthesis of an inventive polyurethaneurea polymer in DMF as solvent. The polymer is identical with that of Example 1, but was prepared in DMF in order to be able to compare its physical properties with the polymer of Example 9.

198.6 g of Desmophen C 2200, 23.0 g of LB 25 and 47.8 g of 4,4′-bis(isocyanatocyclohexyl)methane (H₁₂MDI) were reacted at 110° C. in the melt to a constant NCO content of 2.4%. The product was cooled and diluted with 550 g of DMF. At room temperature a solution of 10.5 g of isophoronediamine in 100 g of 1-methoxypropan-2-ol was added. After the end of the increase in molar weight and the attainment of the desired viscosity range (testing by measurement of the viscosity of a sample taken, using the rheometer cited above) 0.5 g of n-butylamine was added in order to block the small isocyanate content that remained. This gave 930 g of a 30.6% strength solution of polyurethaneurea in DMF with a viscosity of 16 800 mPas at 23° C.

### Example 11

As described in Example 8, films on glass were produced with the polyurethane solutions of Examples 9 and 10, and their static contact angles were measured.

TABLE 2
Static contact angles as a function of the monofunctional
polyethers used

| PU FILM | Contact angle [°] |
|---|---|
| Example 9 (comparative example) | 55 |
| Example 10 (inventive example) | 36 |

The film of Example 10, produced with the mixed (polyethylene oxide/polypropylene oxide) monofunctional polyether alcohol, exhibits, with 36°, a significantly lower static contact angle than the film of Example 9 (55°) that contains pure polyethylene oxide units.

### Example 12

This example describes the synthesis of an inventive polyurethane in organic solution. This product was compared with the polyurethane, prepared correspondingly in aqueous dispersion, of Example 13 (see Example 14).

277.2 g of Desmophen C 2200, 33.1 g of LB 25, 6.7 g of neopentyl glycol, 71.3 g of 4,4′-bis(isocyanatocyclohexyl)methane (H₁₂MDI) and 11.9 g of isophorone diisocyanate were reacted at 110° C. to a constant NCO content of 2.4%. The product was cooled and diluted with 500.0 g of toluene and 350.0 g of isopropanol. At room temperature a solution of 6.2 g of isophoronediamine in 186.0 g of 1-methoxypropan-2-ol was added. After the end of the increase in molar weight and the attainment of the desired viscosity range, the batch was stirred for a further 4 hours in order to block the remaining isocyanate content with isopropanol. This gave 1442 g of a 28.6% strength solution of polyurethaneurea in toluene/isopropanol/1-methoxypropan-2-ol with a viscosity of 17 500 mPas at 23° C.

### Example 13

This example describes the synthesis of the polyurethane of Example 12 in aqueous dispersion. It consists of the same polymer as described in Example 12. The two polymers are compared with one another in Example 14.

277.2 g of Desmophen C 2200, 33.1 g of Polyether LB 25 and 6.7 g of neopentyl glycol were introduced at 65° C. and homogenized by stirring for 5 minutes. Added to this mixture at 65° C. and over the course of 1 minute were, first, 71.3 g of 4,4′-bis(isocyanatocyclohexyl)methane (H₁₂MDI) and thereafter 11.9 g of isophorone diisocyanate. The mixture was heated at 110° C. until a constant NCO value of 2.4% was reached. The completed prepolymer was dissolved at 50° C. in 711 g of acetone and then at 40° C. a solution of 4.8 g of ethylenediamine in 16 g of water was metered in over the course of 10 minutes. The subsequent stirring time was 5 minutes. Subsequently the product was dispersed over the course of 15 minutes by addition of 590 g of water. This was followed by the removal of the solvent by vacuum distillation. This gave a storage-stable polyurethane dispersion having a solids content of 40.7% and an average particle size of 136 nm. The pH of this dispersion was 6.7.

### Example 14

The two coatings of Examples 12 and 13 were applied to release paper using a 200 μm doctor blade. The coating of Example 12 was applied in undiluted form; the aqueous dispersion, before the production of a film, was admixed with 2% by weight of a thickener (Borchi Gel A LA, Borchers, Langenfeld, Germany) and homogenized by stirring at RT for 30 minutes. The wet films were dried at 100° C. for 15 minutes.

The parameters measured are tensile strength and breaking extension in the dry state and after 24 h of water exposure of the films. The investigations were carried out in accordance with DIN 53504.

TABLE 3
Comparison of the tensile strength results for polyurethane from organic
solution and aqueous dispersion

| | | | Breaking | Breaking |
|---|---|---|---|---|
| | Breaking stress | Breaking stress | extension | extension |
| | (N/mm²) | (N/mm²) | (%) | (%) |
| Film | dry film | 24 h water | dry film | 24 h water |
| Example 12 | 25.3 | 24.9 | 700 | 700 |
| Example 13 | 24.8 | 18.3 | 550 | 450 |

The results of the table show that the breaking stress of the dried films corresponds to both polyurethanes, independently of their preparation as a solution or as an aqueous dispersion, within the bounds of experimental accuracy. The film of Example 12 produced from organic solution, however, possesses a higher elasticity (700% breaking extension as compared with 550% for the polymer from aqueous dispersion). Furthermore, breaking stress and breaking extension do not alter, within the bounds of measurement accuracy, for the film produced from organic solution, whereas breaking stress and breaking extension of the film produced from aqueous dispersion decrease significantly.

1.-14. (canceled)

15. A coating composition in the form of a solution comprising at least one polyurethaneurea, wherein the polyurethaneurea is terminated with a copolymer unit comprising polyethylene oxide and polypropylene oxide.

16. The coating composition according to claim 15, wherein the polyurethaneurea comprises units which originate from at least one hydroxyl-containing polycarbonate.

17. The coating composition according to claim 15, wherein the polyurethaneurea comprises units which originate from aliphatic or cycloaliphatic polyisocyanates.

18. The coating composition according to claim 15, wherein the polyurethaneurea comprises units which originate from at least one polyol.

19. The coating composition according to claim 15, wherein the polyurethaneurea comprises units which originate from at least one diamine or amino alcohol.

20. The coating composition according to claim 15, wherein the polyurethaneurea comprises units which originate from further hydroxyl- and/or amino-containing synthesis components.

21. The coating composition according to claim 15, wherein the polyurethaneurea is synthesised from components comprising:
a) at least one polycarbonate polyol;
b) at least one polyisocyanate;
c) at least one monofunctional polyoxyalkylene ether; and
d) at least one diamine or an amino alcohol.

22. The coating composition according to claim 21, wherein the polyurethaneurea synthesis components further comprises at least one polyol.

23. The coating composition according to claim 15, wherein the polyurethaneurea is synthesised from components comprising:
a) at least one polycarbonate polyol having an average molar weight between 400 g/mol and 6000 g/mol and a hydroxyl functionality of 1.7 to 2.3, or mixtures of such polycarbonate polyols;
b) at least one aliphatic, cycloaliphatic or aromatic polyisocyanate or mixtures of such polyisocyanates in an amount per mole of the polycarbonate polyol of 1.0 to 3.5 mol;
c) at least one monofunctional polyoxyalkylene or a mixture of such polyethers, having an average molar weight between 500 g/mol and 5000 g/mol, in an amount per mole of the polycarbonate polyol of 0.01 to 0.5 mol;
d) at least one aliphatic or cycloaliphatic diamine or at least one amino alcohol, as so-called chain extenders, or mixtures of such compounds in an amount per mole of the polycarbonate polyol of 0.1 to 1.5 mol; and
e) optionally, one or more short-chain aliphatic polyols having a molar weight between 62 g/mol and 500 g/mol, in an amount per mole of the polycarbonate polyol of 0.05 to 1.0 mol.

24. A process for preparing a polyurethaneurea solution wherein the polyurethaneurea is terminated with a copolymer unit comprising polyethylene oxide and polypropylene oxide, the process comprising the steps of:
a) reacting the polycarbonate polyol, the polyisocyanate and the monofunctional polyoxyalkylene ether and, optionally, the polyol in the melt or in the presence of a solvent in solution, until all of the hydroxyl groups have been consumed;
b) adding further solvent and, if appropriate, adding the dissolved diamine or the amino alcohol in solution if appropriate; and
c) optionally, blocking the residues of NCO groups which still remain after the target viscosity has been reached, by a monofunctional aliphatic amine.

25. The process according to claim 24, wherein the solvent is selected from the group consisting of N-ethylpyrrolidone, dimethylformamide, N-methylacetamide, tetramethylurea, N-methylpyrrolidone, γ-butyrolactone, aromatic solvent, linear ester, cyclic ester, ether, ketone, alcohol and mixtures thereof.

26. The process according to claim 24, wherein the polyurethane solution has a solids content between 5% to 60% by weight.

27. The coating composition in the form of a solution, obtained by claim 24.

28. A coated medical device which comprises a medical device coated with the coating composition according to claim 15.

29. A process to coat a medical device which comprises coating the medical device with the coating composition according to claim 15.
